(19) United States
(12) Patent Application Publication
(10) Pub. No.: US 2016/0114095 A1
(43) Pub. Date: Apr. 28, 2016

(54) METHODS AND SYSTEMS FOR COLLECTING MONONUCLEAR CELLS

(71) Applicant: Fenwal, Inc., Lake Zurich, IL (US)

(72) Inventor: Katherine N. Radwanski, Des Plaines, IL (US)

(21) Appl. No.: 14/524,115

(22) Filed: Oct. 27, 2014

(51) Int. Cl. A61M 1/36; A61K 35/15; C12N 5/0786

(52) U.S. Cl. CPC A61M 1/3693; C12N 5/0645; A61K 35/15; A61M 1/3681; A61M 2202/0439; A61M 2202/0415

(57) ABSTRACT

A method for obtaining MNCs is set forth. The method includes: separating mononuclear cells from a biological fluid that includes red blood cells, plasma and platelets and collecting a targeted number of mononuclear cells in a suspension including plasma and residual red blood cells and platelets; concentrating the separated mononuclear cells; removing plasma from the concentrated mononuclear cells until the amount of residual plasma remaining with the concentrated mononuclear cells reaches a pre-determined volume; and adding a crystalloid solution to the concentrated mononuclear cells. Related apparatus and resultant MNC products are also disclosed.

### FIELD OF THE DISCLOSURE

The present disclosure is directed to systems and methods for collecting mononuclear cells. More particularly, the present disclosure is directed to systems and methods for collecting mononuclear cells with a reduced volume of residual plasma so as to reduce plasma interference during photopheresis.

### BACKGROUND

Whole blood is made up of various cellular components such as red cells, white cells (or leukocytes) and platelets suspended in its liquid component, plasma. Whole blood can be separated into its constituent components (cellular or liquid), and the desired component can be separated so that it can be administered to a patient in need of that particular component. For example, mononuclear cells (MNCs), primarily lymphocytes and monocytes, can be removed from the whole blood of a patient, collected, and subjected to photodynamic therapy in a procedure commonly referred to as extracorporeal photopheresis, or ECP. In ECP, MNCs are treated with a photosensitizing agent and subsequently irradiated with specified wavelengths of light to achieve a desired effect, and returned to the patient for the treatment of various blood diseases to, e.g., eliminate immunogenicity in cells, inactivate or kill selected cells, inactivate viruses or bacteria, or activate desirable immune responses.

More specifically, separated MNCs may be separated from whole blood, chemically treated with a light-activated agent, such as 8-methoxypsoralen (8-MOP, either added to the separated MNCs or administered in advance to the patient), exposed to ultraviolet light, and returned to the patient. The activated 8-methoxypsoralen crosslinks with the DNA in the exposed MNCs, ultimately resulting in apoptosis of the MNCs. The photochemically-damaged MNCs returned to the patient to induce cytotoxic effects on T-cell formation, resulting in an antitumor action.

Commonly, MNCs are collected by introducing whole blood into a centrifuge chamber wherein the whole blood is separated into its constituent components based on the size and densities of the different components, with the targeted components) being collected, and the non-target components either being returned to the patient or otherwise disposed of. Typical blood processing systems thus include a permanent, reusable centrifuge assembly containing the hardware (drive system, pumps, valve actuators, programmable controller, and the like) that spins and pumps the blood, and a disposable, sealed and sterile fluid processing assembly that is mounted cooperatively on the hardware. The centrifuge assembly spins a disposable centrifuge chamber in the fluid processing assembly during a collection procedure, thereby separating the blood into its constituent components.

A difficulty in performing phototherapy is the delivery of the proper dose of light energy to the photoactivatable material in the suspension, particularly if the suspension includes material that is not substantially transparent to light so that it attenuates the light energy intended for photoactivation. Specifically, during the harvest of MNCs for photopheresis, other non-target substances are present in the MNC product that can interfere with delivering the intended dose of UV-A light to the target MNCs. Red blood cells absorb the majority of UV-A light in MNCs products. However, plasma proteins and lipids can also contribute to UV-A light absorption, especially in the case of certain disease states or medications, such as elevated bilirubin levels and drugs such as mycophenolate mofetil (MMF) and cyclosporine, which cause hyperlipidemia.

By way of the present disclosure, systems and methods are provided for collecting mononuclear cells with a reduced volume of residual plasma so as to reduce plasma interference during photopheresis.

### SUMMARY

In one aspect, the present disclosure is directed to a method for obtaining mononuclear cells and preparing the mononuclear cells for photopheresis with a separation device. The method comprises: separating mononuclear cells from a biological fluid that includes red blood cells, plasma and platelets and collecting a targeted number of mononuclear cells in a suspension including plasma and residual red blood cells and platelets; concentrating the separated mononuclear cells; removing plasma from the concentrated mononuclear cells until the amount of residual plasma remaining with the concentrated mononuclear cells reaches a pre-determined volume; and adding a crystalloid solution to the concentrated mononuclear cells.

In another aspect of the method, the volume of residual plasma remaining with the concentrated mononuclear cells may be less than or equal to (5) 50 mL.

In another aspect, the concentrated mononuclear cells may have a hematocrit of less than or equal to (≦) 2%, after adding the crystalloid solution.

In a further aspect of the method, crystalloid solution may be added to the concentrated mononuclear cells in an amount so that the volume % of plasma is less than or equal to (≦) 25%.

In a related aspect, the amount of crystalloid solution may be added to the suspension in an amount of from 150 mL to 200 mL.

In another aspect of the method, the separation device may be flushed with crystalloid solution or plasma.

In another aspect, the present disclosure is directed to a system for the collection of mononuclear cells. The system includes a reusable hardware apparatus with a separation device and a programmable microprocessor driven controller including instructions for processing a biological fluid. The system also includes a disposable processing circuit associated with the apparatus, wherein the circuit includes a processing chamber for receiving a biological fluid and one or more containers for collecting mononuclear cells. The controller is programmed to separate mononuclear cells from a biological fluid that includes red blood cells, plasma and platelets, concentrate the mononuclear cells and remove plasma until the residual plasma remaining with the concentrated mononuclear cells reaches a pre-determined volume, and combine the concentrated mononuclear cells with a crystalloid solution.

In another aspect, the present disclosure is directed to a mononuclear cell product that includes mononuclear cells, plasma, crystalloid solution and residual red blood cells and platelets having a total volume of less than or equal to (≦) 200 mL and a hematocrit of less than or equal to (≦) 2%. In a related aspect, the product may have a volume % of plasma of less than or equal to (≦) 25% and/or a volume of residual plasma of less than or equal to (≦) 50 mL.

### BRIEF DESCRIPTION OF THE DRAWINGS

FIG. 1 is a is a perspective view of an automated apheresis device that may be used in the collection and other processing steps of mononuclear cells in accordance with the present disclosure;

FIG. 2 is an enlarged perspective view of the front panel of the device of FIG. 3 with an exemplary disposable processing set for collecting mononuclear cells mounted on the device;

FIG. 3 is a diagram showing the disposable processing set of FIG. 2;

FIG. 4 is a graph showing lymphocyte apoptosis levels during 72 hours of culture post ECP treatment (30 J/cm²) of MNC products (200 mL, 2% Hct) suspended in predominantly plasma or saline solution; and

FIG. 5 is a graph showing standard curves generated from purified MNCs in saline treated with known UV-A light doses ranging from 0-2.0 J/cm².

### DETAILED DESCRIPTION OF THE EMBODIMENTS

A more detailed description of the systems and methods in accordance with the present disclosure is set forth below. It should be understood that the description below of specific devices and methods is intended to be exemplary, and not exhaustive of all possible variations or applications. Thus, the scope of the disclosure is not intended to be limiting, and should be understood to encompass variations or embodiments that would occur to persons of ordinary skill.

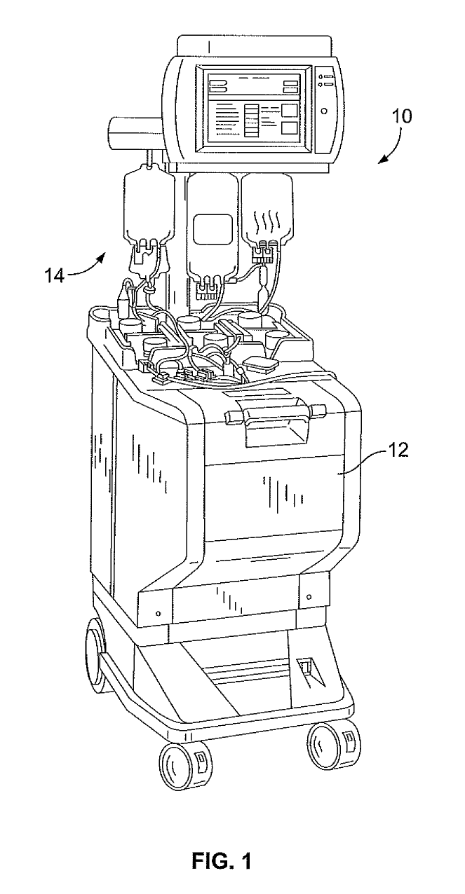

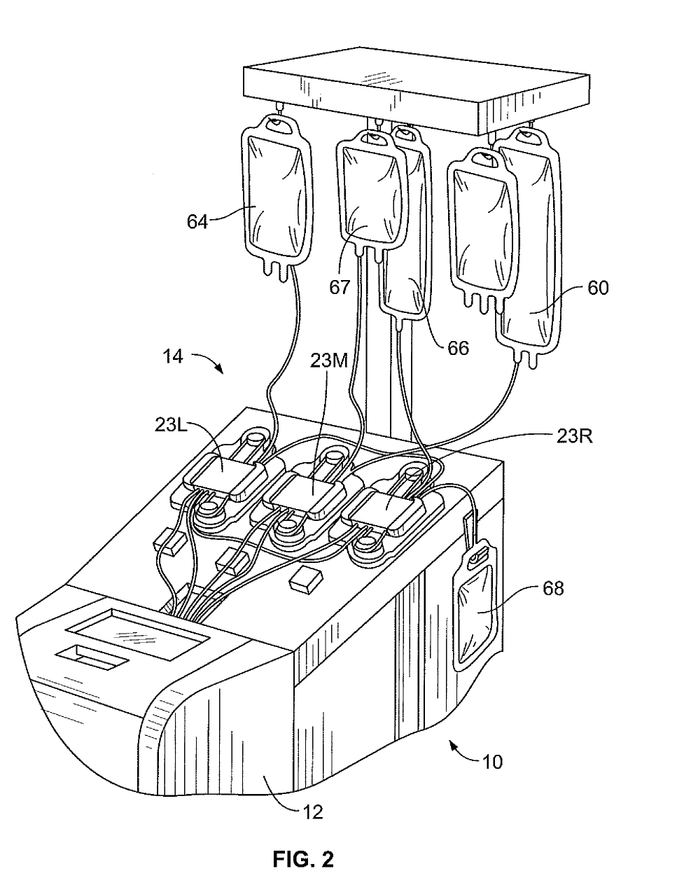

FIGS. 1 and 2 show a representative separation system 10 useful in the separation and collection of mononuclear cells, as described herein. The system 10 includes a durable separation component 12 and a disposable processing kit 14 that is mounted thereon. In one embodiment, the separation principle used by the separator 12 is based on centrifugation, but an automated separator based on a different separation principle may also be used.

When used to perform photopheresis, an irradiation component housed separately from separation component is used in conjunction with the separation system 10, as shown and described in US 2013/0197419, which is incorporated herein by reference. Although separately housed and independent devices, it is preferable that separation device and irradiation device be located adjacent to each other. However, it will be appreciated that the methods described herein may also be used with devices having integrated separation and irradiation components.

In accordance with the systems and methods described herein a patient is connected to a blood processing set, i.e., fluid circuit 14. The fluid circuit 14 provides a sterile closed pathway between the separation component 12 and the irradiation component. The system described herein also optionally includes a washing component which, preferably, is housed within the separation component 12. Preferably, the separation component 12 and washing component are one and the same.

With reference to FIG. 1, whole blood is withdrawn from the patient and introduced into the separation component 12, where the whole blood is separated to provide a target cell population, which in the context of the present disclosure may be mononuclear cells. Other components separated from the whole blood, such as red blood cells and platelets may be returned to the patient or collected in pre-attached containers of the blood processing set. The separated target cell population, e.g., mononuclear cells, is then prepared for treatment in accordance with the methods described in greater detail below.

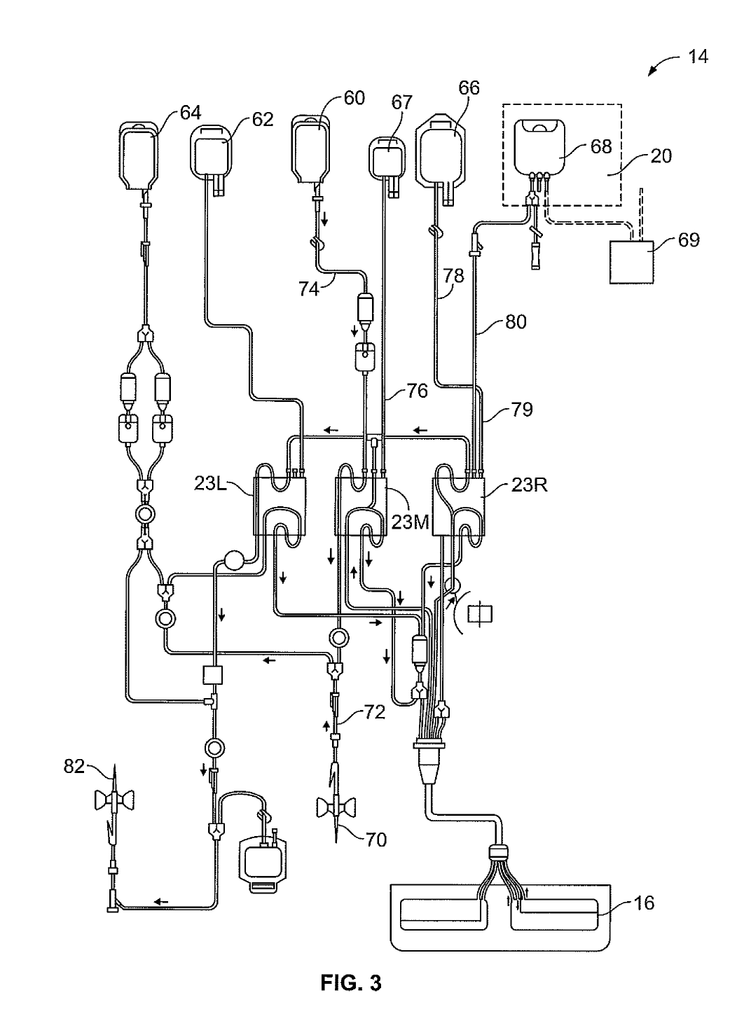

Apparatus useful in the collection (and washing) of mononuclear cells, and providing the separation component 12 of FIG. 1, include the Amicus Separator made and sold by Fenwal, Inc., of Lake Zurich, Ill. Mononuclear cell collections using a device such as the Amicus® are described in greater detail in U.S. Pat. No. 6,027,657, the contents of which is incorporated by reference herein in its entirety. The fluid circuit (FIG. 3) includes a blood processing container 16 defining a separation chamber suitable for harvesting mononuclear cells (MNC) from whole blood.

As shown in FIG. 2, a disposable processing set or fluid circuit 14 (which includes container 16) is mounted on the front panel of the separation component 12. The processing set (fluid circuit 14) includes a plurality of processing fluid flow cassettes 23L, 23M and 23R with tubing loops for association with peristaltic pumps on separation component 12. Fluid circuit 14 also includes a network of tubing and pre-connected containers for establishing flow communication with the patient and for processing and collecting fluids and blood and blood components, as shown in greater detail in FIG. 3.

As seen in FIG. 3, the disposable processing set 14 may include a container 60 for supplying anticoagulant, a waste container 62 for collecting waste from one or more steps in the process for treating and washing mononuclear cells, a container 64 for holding a crystalloid solution, such as saline, or other wash or resuspension medium, a container 66 for collecting plasma, a container 68 for collecting the mononuclear cells and, optionally, container 69 for holding photoactivation agent.

Container 68 may also serve as the illumination container, and is preferably pre-attached to with the disposable set 14. Alternatively, container 68 may be attached to set 14 by known sterile connection techniques, such as sterile docking or the like.

With reference to FIG. 3, fluid circuit includes inlet line 72, an anticoagulant (AC) line 74 for delivering AC from container 60, an RBC line 76 for conveying red blood cells from chamber 12 of container 14 to container 67, a platelet-poor plasma (PPP) line 78 for conveying PPP to container 66 and line 80 for conveying mononuclear cells to and from separation chamber 16 and collection/illumination container 68.

The blood processing set also includes one or more venipuncture needle(s) for accessing the circulatory system of the patient. As shown in FIG. 3, fluid circuit 14 includes inlet needle 70 and return needle 82. In an alternative embodiment, a single needle can serve as both the inlet and outlet needle.

Fluid flow through fluid circuit 14 is preferably driven, controlled and adjusted by a microprocessor-based controller in cooperation with the valves, pumps, weight scales and sensors of separation component 12 and fluid circuit 14, the details of which are described in the previously mentioned U.S. Pat. No. 6,027,657.

The illustrated fluid circuit is further adapted for association with the treatment component (i.e., irradiation device). Apparatus for the irradiation of the mononuclear cells are also known and are available from sources such as Cerus Corporation, of Concord, Calif. One example of a suitable irradiation device is described in U.S. Pat. No. 7,433,030, the contents of which are likewise incorporated by reference herein in its entirety. As shown and described in U.S. Pat. No. 7,433,030, irradiation device preferably includes a tray or other holder for receiving one or more containers during treatment. Other irradiation devices may also be suitable for use with the method and system described herein, including devices available from Macopharma and/or Vilber Lourmet.

A separation chamber is defined by the walls of the processing container 16. In operation, the separation device 12 rotates the processing container 16 about an axis, creating a centrifugal field within the processing container 16. Details of the mechanism for causing of the processing container 16 are disclosed in U.S. Pat. No. 5,360,542 entitled “Centrifuge with Separable Bowl and Spool Elements Providing Access to the Separation Chamber,” which is also incorporated herein by reference.

In one embodiment, an apheresis device may include a programmable controller that is pre-programmed with one or more selectable protocols. A user/operator may select a particular processing protocol to achieve a desired outcome or objective. The pre-programmed selectable protocol(s) may be based on one or more fixed and/or adjustable parameters. During a particular processing procedure, the pre-programmed controller may operate the centrifuge and processing chamber associated therewith to separate blood into its various components, as well as operate one or more pumps to move blood, blood components and/or solutions through the various openable valves and tubing segments of a processing set, such as such as processing set 14 illustrated in FIG. 3. This may include, for example, initiating and causing the centrifugal separation of mononuclear cells from whole blood in the separation chamber 16, removing plasma from mononuclear cells (i.e., pumping the removed plasma to a storage or waste bag) to obtain MNC concentrate, purging or flushing the tubing segments to collect additional MNCs that may reside or remain in the tubing during or after processing, and combining a crystalloid solution to the concentrated MNCs. The various processing steps performed by the pre-programmed automated apheresis device may occur separately, in series, simultaneously or any combination of these.

In accordance with the present disclosure, an automated apheresis device may be used to perform MNC collection in a batch process in which MNCs continuously collect in the chamber 16 until the target cycle volume is reached. As the MNCs are transferred out of the chamber 16, they pass through an optical sensor which detects the presence of cells in the tubing line to determine the start and end of the MNC harvest (i.e. when to open and close the valves leading to the product container). After MNC harvest is complete, the remaining cells in the line are flushed into the product container with a predetermined volume of plasma known as the “plasma flush”. The plasma volume could be further reduced by reducing, eliminating or replacing the plasma flush with a crystalloid solution flush.

The volume of the MNC product (which contains MNCs, RBCs and platelets in plasma) is fixed at a low predetermined value. If needed, additional cycle(s) of WB processing and MNC harvest can be performed to achieve the desired yield of MNCs to be treated. Most of the plasma is removed from the MNC concentrate to arrive at a predetermined volume of residual plasma, and the MNC concentrate is then reconstituted with a crystalloid solution to obtain a MNC product ready for phototherapy. More specifically, sufficient crystalloid solution (preferably saline) is added to the concentrated MNCs to obtain the desired hematocrit for UV-A irradiation (typically around 150-200 mL of saline is added to achieve a hematocrit of 2% or less). As the plasma content in the overall product represents typically 25% or less of the total volume, the effect of elevated plasma protein or lipid levels on UV-A dose delivered to MNCs is reduced.

Experimentation was performed to confirm the efficacy of the present method in reducing plasma interference during ECP. This was done by comparing lymphocyte apoptosis levels 72 hours after UV-A exposure for ECP-treated MNC products suspended in plasma versus ECP-treated MNC products suspended in a crystalloid solution (i.e., saline).

MNC products were collected from healthy donors using the following collection settings: 1cycle, WB/cycle=2000 mL, plasma flush=10 mL, and plasma storage fluid=150 mL or 0 mL. MNC products were suspended in approximately 220 mL volume in order to provide 20 mL samples for study controls (+8MOP, no UV). For MNC products collected with 150 mL plasma storage fluid, approximately 30 mL of saline was added to the product post collection using a sterile syringe (plasma arm, n=6). For MNC products collected with 0 mL storage fluid, approximately 190 mL of saline was added to the product post collection using a sterile syringe (saline arm, n=6).

8-MOP was added to each MNC product to a final concentration of 200 ng/mL and products (200 mL, ˜2% Hct) were irradiated with 30 J/cm² UV-A light (320-400 nm) with an intensity of 13-20 J/cm². After irradiation, MNCs were purified using a Ficoll-Paque gradient, washed twice with RPMI 1640 media, and re-suspended at 1-2×10⁶/mL in RPMI 1640 media supplemented with 2 mM glutamine and 10% human serum.

Cells were cultured at 37° C. in a humidified chamber with 5% CO₂ for up to 72 hours. After 24, 48 and 72 hours, samples were assayed for apoptosis. Lymphocyte apoptosis was measured as the percentage of CD45+/Annexin-V positive cells in the lymphocyte forward/side scatter gate.

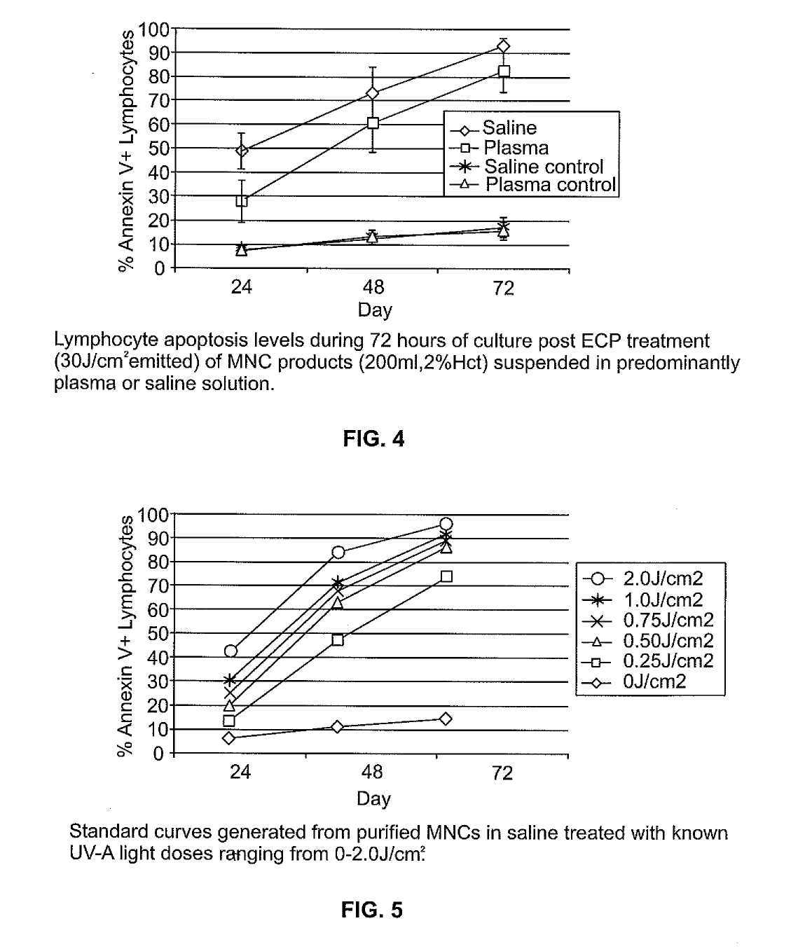

The results are plotted in FIGS. 4 and 5. During culture, treated lymphocytes in the plasma arm of the study exhibited an apoptotic response similar to 0.5-1.0 J/cm² delivered to the MNCs, while the saline arm was similar to 1.5-2.0 J/cm². It was thus concluded that using primarily saline solution in the MNG product versus plasma resulted in at least 2 times greater UV-A light dose delivered to the MNCs.

### EXAMPLES

Without limiting any of the foregoing, the subject matter described herein may be found in one or more methods, systems and/or products. For example, in a first aspect of the present subject matter a method for obtaining MNCs is set forth. The method includes: separating mononuclear cells from a biological fluid that includes red blood cells, plasma and platelets and collecting a targeted number of mononuclear cells in a suspension including plasma and residual red blood cells and platelets; concentrating the separated mononuclear cells; removing plasma from the concentrated mononuclear cells until the amount of residual plasma remaining with the concentrated mononuclear cells reaches a pre-determined volume; and adding a crystalloid solution to the concentrated mononuclear cells.

In another aspect of the method, the volume of residual plasma remaining with the concentrated mononuclear cells may be less than or equal to (≦) 50 mL.

In another aspect, the concentrated mononuclear cells may have a hematocrit of less than or equal to (≦) 2%, after adding the crystalloid solution.

In a further aspect of the method, crystalloid solution may be added to the concentrated mononuclear cells in an amount so that the volume % of plasma is less than or equal to (≦) 25%.

In a related aspect, the amount of crystalloid solution may be added to the suspension in an amount of from 150 mL to 200 mL.

In another aspect of the method, the separation device may be flushed with crystalloid solution or plasma.

In another aspect, the present disclosure is directed to a system for the collection of mononuclear cells. The system includes a reusable hardware apparatus with a separation device and a programmable microprocessor driven controller including instructions for processing a biological fluid. The system also includes a disposable processing circuit associated with the apparatus, wherein the circuit includes a processing chamber for receiving a biological fluid and one or more containers for collecting mononuclear cells. The controller is programmed to separate mononuclear cells from a biological fluid that includes red blood cells, plasma and platelets, concentrate the mononuclear cells and remove plasma until the residual plasma remaining with the concentrated mononuclear cells reaches a pre-determined volume, and combine the concentrated mononuclear cells with a crystalloid solution.

In another aspect, the present disclosure is directed to a mononuclear cell product that includes mononuclear cells, plasma, crystalloid solution and residual red blood cells and platelets having a total volume of less than or equal to (≦) 200 mL and a hematocrit of less than or equal to (≦) 2%. In a related aspect, the product may have a volume % of plasma of less than or equal to (≦) 25% and/or a volume of residual plasma of less than or equal to (≦) 50 mL.

1. A method for obtaining mononuclear cells and preparing the mononuclear cells for photopheresis with a separation device comprising:
a) separating mononuclear cells from a biological fluid that includes red blood cells, plasma and platelets;
b) collecting a targeted number of mononuclear cells in a suspension including plasma and residual red blood cells and platelets;
c) concentrating said separated mononuclear cells;
d) removing plasma from the concentrated mononuclear cells until the amount of residual plasma remaining with the concentrated mononuclear cells reaches a pre-determined volume; and
e) adding a crystalloid solution to the concentrated mononuclear cells.

2. The method of claim 1 wherein said volume of residual plasma remaining with said concentrated mononuclear cells is less than or equal to (≦) 50 ml.

3. The method of claim 1 wherein the concentrated mononuclear cells after adding the crystalloid solution has a hematocrit of less than or equal to (≦) 2%.

4. The method of claim 1 wherein crystalloid solution is added to the concentrated mononuclear cells so that the volume % of plasma is less than or equal to (≦) 25%.

5. The method of claim 1 in which from 150 mL to 200 mL of crystalloid solution is added to the suspension.

6. The method of claim 1 further comprising collecting said mononuclear cells in a collection container suitable for irradiation of the mononuclear cells.

7. The method of claim 1 comprising separating and concentrating said mononuclear cells in one or more chambers of a processing apparatus.

8. The method of claim 7 comprising subjecting said processing apparatus to a centrifugal field sufficient to separate and concentrate said mononuclear cells.

9. The method of claim 1 wherein said biological fluid comprises whole blood.

10. The method of claim 1 wherein the suspension comprises a quantity of mononuclear cells recovered from separation device by flushing the separation device.

11. The method of claim 10 wherein the separation device is flushed with a crystalloid solution.

12. The method of claim 10 wherein the separation device is flushed with plasma.

13. The method of claim 11 wherein the separation device is flushed with 10 mL of crystalloid solution.

14. The method of claim 12 wherein the separation device is flushed with 10 mL of plasma.

15. A system for the collection of mononuclear cells comprising:
a) a reusable hardware apparatus, said apparatus including a separation device and a programmable microprocessor driven controller including instructions for processing a biological fluid;
b) a disposable processing circuit associated with said apparatus, said circuit including a processing chamber for receiving a biological fluid and one or more containers for collecting mononuclear cells;
c) wherein said controller is programmed to:
i. separate mononuclear cells from a biological fluid that includes red blood cells, plasma and platelets;
ii. concentrate said obtained mononuclear cells and remove plasma from the concentrated mononuclear cells until the amount of residual plasma remaining with the concentrated mononuclear cells reaches a pre-determined volume; and
iii. combine said obtained platelets with a crystalloid solution.

16. The system of claim 15 wherein said separation device comprises a rotatable element.

17. The system of claim 15 wherein said rotatable element generates a centrifugal field sufficient to separate mononuclear cells from other components of a biological fluid.

18. The system of claim 15 further comprising a source of a crystalloid solution.

19. The system of claim 15 wherein said controller is programmed to deliver a selected volume of said crystalloid solution to concentrated mononuclear cells in said residual amount of plasma.

20. A mononuclear cell product comprising mononuclear cells, plasma, crystalloid solution and residual red blood cells and platelets having a total volume of less than or equal to (≦) 200 mL and a hematocrit of less than or equal to (≦) 2%, wherein the volume % of plasma is less than or equal to (≦) 25% and the volume of residual plasma remaining with said concentrated mononuclear cells is less than or equal to (≦) 50 ml.

21. (canceled)

22. (canceled)
